(19) United States
(12) Patent Application Publication
(10) Pub. No.: US 2009/0216134 A1
(43) Pub. Date: Aug. 27, 2009

(54) SPHYGMOMANOMETER

(75) Inventors: Stefan Hollinger, Kronberg (DE); Dieter Wunder, Schotten (DE); Ulrich Heck, Krefeld (DE); Dirk Freund, Kelkheim (DE); Brigitte Harttmann, Niedernhausen (DE); Gerrit Roenneberg, Darmstadt (DE); Fred Schnak, Kronberg (DE)

Correspondence Address: DARBY & DARBY P.C., P.O. BOX 770, Church Street Station, New York, NY, 10008-0770, US

(73) Assignee: Kaz USA, Inc., Southborough, MA (US)

(21) Appl. No.: 11/912,408

(22) PCT Filed: Mar. 10, 2006
(86) PCT No.: PCT/EP06/02204

§ 371 Date: Dec. 1, 2008

(30) Foreign Application Priority Data
Apr. 28, 2005 (DE) 10 2005 019 755.8

(51) Int. Cl. A61B 5/02

(52) U.S. Cl. 600/496

(57) ABSTRACT

The invention relates to a sphygmomanometer for measuring blood pressure over a long period of time, especially over 24 hours, with a cuff which can be placed against an extremity of the body, a measuring device for determining blood pressure data, a control device for activating automatically the measuring device at specified time intervals, as well as a memory device for storing the blood pressure data determined in different measurements. Pursuant to the invention, the sphygmomanometer is novel owing to the fact that it is constructed as a portable instrument, for which the measuring device, a control device and the memory are part of the body of the device, which is fastened at the cuff.

### CROSS-REFERENCE TO PRIOR APPLICATION

This is a U.S. National Phase application under 35 U.S.C § 371 of International Application No. PCT/EP2006/002204, filed Mar. 10, 2006, and claims the benefit of German Patent Application No. 10 2005 019 755.8 filed Apr. 28, 2005, both of which are incorporated herein. The International Application was published in German on Nov. 2, 2006 as WO 2006/114157 A1 under PCT Article 221 (2).

### BACKGROUND OF THE INVENTION

1. Field of the Invention

The present invention relates to a sphygmomanometer for measuring blood pressure over a long period, especially over 24 hours, with a cuff which can be placed against an extremity of the body, a measuring device for determining blood pressure data, a control device for activating automatically the measuring device at specified time intervals, as well as a memory device for storing the blood pressure data determined in different measurements.

2. Description of the Related Art

24-hour sphygmomanometers are valuable instruments for medical diagnosis of arterial hypertension. With these instruments, the blood pressure is measured automatically according to a specified time grid in the course of one day and one night. The blood pressure data obtained is stored in a memory and read by the physician at the end of the measurement period for statistical processing and evaluation.

Conventional sphygmomanometers, which measure blood pressure over a prolonged period, such as those known from U.S. Pat. No. 6,699195 B2, usually are very expensive instruments, which must be lent to the patient by a physician in the course of the treatment. For reasons of costs however, physicians are not willing or not able to keep a larger number of these 24-hour measuring instruments ready. Unfortunately, this leads to the situation in which the physician is unable to carry out 24-hour measurements in the scope regarded as meaningful by him and to the required extent.

A further limitation arises out of the fact that the handling of conventional, commercial sphygmomanometers for measurements over a long period unfortunately is frequently rather cumbersome, so that the procedure over a whole day is regarded as tiresome and the patients refuse to participate in a meaningful second 24-hour measurement. The inconvenient handling arises especially owing to the fact that mobile 24-hour sphygmomanometers must be worn on a at the belt and at the measuring site and with a cable connecting the two components. This is very inconvenient, especially during the night.

Overall, this leads to the situation that far fewer 24-hour blood pressure analyses are carried out than are medically meaningful or even necessary.

### SUMMARY OF THE INVENTION

The present invention provides a remedy. The object of the invention is to make available an improved sphygmomanometer, which, within a wide framework, makes blood pressure measurements which extend over a long time both cost effective and simple.

Pursuant to the invention, this objective is accomplished by the sphygmomanometer of claim 1. Preferred embodiments of the invention are the object of the appended claims.

Pursuant to the invention, the sphygmomanometer is constructed as a portable instrument, for which the measuring device, the control device and the memory device are part of the instrument, and are fastened to the cuff. In contrast to the prior art of U.S. Pat. No. 6,699,195 B2, the cuff, with the measuring apparatus provided thereon, need not be connected to a table instrument for each measurement for evaluation and storage. Instead, the whole of the sphygmomanometer, together with all components, which are necessary for measurements over a prolonged period, can be placed against the respective limb of the body together with the cuff, so that the patient is hampered significantly less by the measuring instrument. The basic concept of the invention is the utilization of a sphygmomanometer, which a patient with a prior history of hypertension usually owns in any case, for the 24-hour measurement. This avoids further expenditures and an equipment bottleneck on the part of the physician, since the latter need not have these instruments available in his practice. Moreover, such domestic instruments are clearly less expensive than the 24-hour instruments of classic coinage.

In a further development of the invention, the body of the sphygmomanometer has an interface, to which can be connected an insertable and removable memory card for long-time measurements, especially a chip card in the form of a SmartCard and/or a memory card. By these means, the sphygmomanometer can be expanded by inserting the memory card for long-time measurements. The basic instrument, as such, may be constructed as a conventional domestic instrument. By equipping it with an additional module, it may be adapted, even subsequently, for long-time measurements in order to equip the control device of the instrument for measurements conducted over a long time and to make available memory capacity for storing the plurality of measurement data. Due to this modular construction, the basic instrument is not burdened by superfluous costs. Moreover, by inserting different memory cards, different long-time measuring strategies can be realized in a simple manner.

The memory card may perform different functions. According to one embodiment of the invention, the memory card forms, at least partly, the memory device for storing blood pressure data. In addition or alternatively to a construction strictly as a memory card, the control device for automatically activating the measuring device at specifiable time intervals may also be provided at least partly on the insertable card. The control means for controlling the measuring device, stored appropriately on the memory and/or chip card, may advantageously interact with the basic control, which is provided permanently in the body of the instrument and which the basic instrument uses for carrying out conventional single measurements.

In a further development of the invention, in order to keep the operation of the instrument simple, the instrument may be switched over automatically to the long-time measurement mode when the memory card is inserted. The control device and/or the memory card may have suitable control means, which, by inserting the memory card in the body of the instrument, permits the sphygmomanometer to conducts measurements over a long time and/or activates it and/or equips it technically with controls.

Advantageously, the control device and/or the memory card may be programmed with control commands for measurements extending over a long time. In particular, the physician can pre-program the operating parameters of the long-time measurements, such as the measurement intervals, by these means. The sphygmomanometer may have input interfaces, through which the control device can be programmed for programming the specifiable parameters of the long-time measurement. Alternatively or additionally, the memory card can be taken out of the sphygmomanometer for this purpose and inserted in a write device.

The data obtained during a long-time measurement can be read either in the physician's office or after being transmitted. especially over a telephone line or the Internet. For this purpose, the sphygmomanometer may have a reading interface, over which the measurement data, stored in the memory, can be read. Alternatively or in addition, the memory card may be taken out of the instrument for this purpose and inserted in a reader.

In a further development of the invention, the control device of the sphygmomanometer differentiates between day and night measurements and carries these out differently and/or, together with the blood pressure data, stores further data, which permits the respective measurement to be identified as a day or a night measurement.

The control device may comprise, in particular, a day control for carrying out measurements during the day and a night control for carrying out measurements at night, by means of which different measurement procedures, depending on the time of day and on parameters, identifying day or night measurements, can be carried out.

Particularly for day measurements, the patient can be urged to bring the sphygmomanometer into a nominal position, especially level with the heart, for the respective measurement and to avoid movements, in order to increase the precision of the measurement. In a further development of the invention, the sphygmomanometer may have an alarm device for this purpose, which emits an alarm signal before a measurement is carried out. Preferably, only the day control of the control device contains means for triggering the alarm device, so that the latter is activated only for day measurements and does not disturb the sleep of the patient.

The measuring device can be controlled by the night control so that it develops less noise. In particular, the pump for filling and/or emptying the cuff of the sphygmomanometer can be operated with less power by the night control, in order to reduce noise.

In a further development of the invention, the sphygmomanometer may have a position detecting device, especially a tilt, motion and/or acceleration sensor, for determining the position of the sphygmomanometer relative to the heart of the patient. The position data may be supplied to the control device, so that the latter can detect and even compensate for deviations from nominal position during the determination of blood pressure data and/or of artifacts resulting from undesirable movement during the determination of blood pressure data. Alternatively or additionally, the position data can be stored together with the blood pressure data determined in the memory device, so that, during the subsequent evaluation of the stored blood pressure data, the effect of the faulty position and/or movements can be evaluated.

The parameters, determined with the help of the position-determining and/or movement-detecting device, can also be used for changing the measuring process. In a further development of the invention, the day control of the controlling device can compare the actual value of the position data and/or the motion data with nominal values and, depending on this comparison, initiate a measurement only if the sphygmomanometer is held sufficiently closely to the nominal position and/or sufficiently few movements are carried out. Alternatively or additionally, the day control system may also work as a function of the alarm signal of the aforementioned alarm device and carry out a measurement only at the end of a specified time span after the alarm signal is emitted.

In a further development of the invention, the control device switches over between its day control and night control as a function of the position-determining and/or movement-detecting device. The night or resting phase can be recognized owing to the fact that relatively few movements are carried out and/or the sphygmomanometer is held relatively constantly level with the heart.

Alternatively or additionally, the night control of the control device may work independently of the signals of the position-determining and/or movement-detecting device. For measurements at night, it may be assumed, that when a patient is lying down, there are no positioning problems with regard to the position of the sphygmomanometer relative to the heart.

### BRIEF DESCRIPTION OF THE DRAWINGS

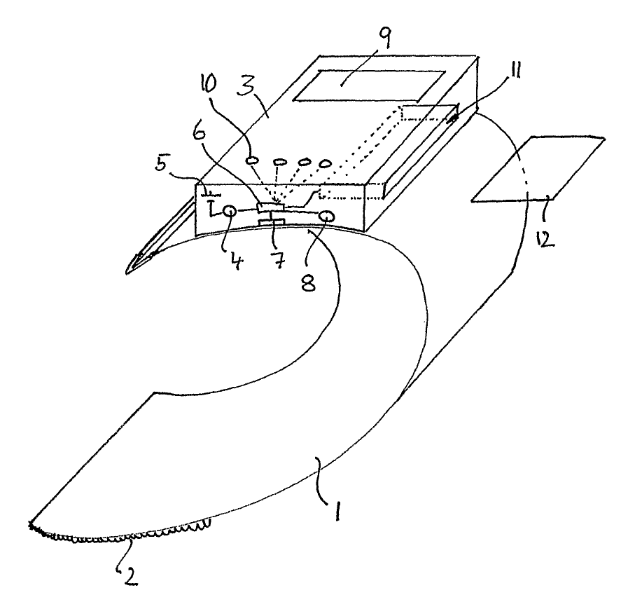

These and further distinguishing features are evident from the claims as well as from the following description and drawings. The individual distinguishing features can be realized by themselves or in the form of sub-combinations for an embodiment of the invention and also in other fields. An example of the invention is shown in a drawing and explained in greater detail in the following. In the drawing, FIG. 1 shows a diagrammatic, perspective view of a wrist sphygmomanometer according to a preferred embodiment of the invention, which can be expanded by means of an insertable memory card for measurements over a long period.

### DETAILED DESCRIPTION OF THE INVENTION

The sphygmomanometer shown in FIG. 1 comprises a cuff 1, which can be placed about a wrist of a patient and can be fixed there, for example, by means of a positive hook fastener 2. The cuff 1 can be filled in a known manner with a fluid. For this purpose, a pump 4 is provided in the equipment housing 3, which is fastened to the upper side of the cuff 1. The pump 4 is supplied from an energy source 5, for example, in the form of batteries or an accumulator, which can be accommodated in an appropriate compartment in the equipment housing 3. The pump 4 is controlled by a control device 6, which is also accommodated in the equipment housing 3, so that the cuff 1 can be blown up and then vented once again according to a specified course.

Furthermore, the sphygmomanometer comprises a pressure sensor 7, with the help of which pressure signals in the cuff 1 generated during a measuring process can be detected and then evaluated by the control device 6. Furthermore, a position and motion sensor 8 is provided in the equipment housing 3. By means of a signal from this sensor 8, the control device 6 can recognize whether the sphygmomanometer is held sufficiently close to its nominal position at the heart and sufficiently steadily.

As shown in FIG. 1, a display 9 for displaying the blood pressure measured and/or for indicating signals or commands to the user, as well as several control keys 10 for inputting control commands, both of which are connected with the control device 6, are provided at the equipment housing 3.

Furthermore, the equipment housing 3 comprises an interface 11, into which a memory card 12, in the form of a chip card and/or a memory card, can be inserted, with the help of which the sphygmomanometer can be expanded into a long-time measurement instrument. On the one hand, the memory card 12 forms a writeable storage device and makes adequate memory locations available for storing the measurement data obtained during the long-time measurements. On the other hand, a special control algorithm, which interacts with the control device 6 in the housing 3 of the equipment and adapts the control device 6 for carrying out long-time measurements, may be stored on the memory card. In particular, the long-time measurement procedure, especially the intervals between measurements, according to which the control device 6 automatically activates the pumps 4 and, with the help of the pressure sensor 7 and of the position and motion sensor 8, carries out a measurement, may be stored on the memory card 12.

The insertion slot for the chip card in the housing can also, alternatively, be constructed at one of the three end faces, other than the one shown here, that is, in particular, not at the side shown, at which the housing is held at the chest for the measurement.

The memory card 12 may be pre-programmed with the necessary control commands by the physician. If the memory card 12 is inserted into the instrument housing 3, the long-time mode of the control device 6 is activated automatically. At the conclusion of the measurement, the measurement data, written during the long-time measurement on the memory card 12, can be evaluated by the physician. For this purpose, the memory card 12 can be removed once again from the instrument housing 3 and inserted into an appropriate reader. Alternatively or in addition, an appropriate interface, over which the data from the control device 6 and the inserted memory card 12 can be read or written in the reverse direction, may also be provided at the instrument housing 3.

1. A sphygmomanometer for measuring blood pressure over a period of at least about 24 hours, comprising:
a cuff, configured to be, placed against an extremity of the body,
a measuring device for determining blood pressure,
a control device for automatically activating said measuring device at specified predetermined time intervals,
a memory device for storing a plurality of blood pressure measurements and
a common housing, said measuring device, said control device, and said memory device being provided within said common housing,
wherein said sphygmomanometer is portable, and
wherein said common housing is disposed on said cuff.

2. The sphygmomanometer of claim I wherein said extremity is a wrist.

3. The sphygmomanometer of claim 1, wherein said common housing comprises an interface for connecting an insertable and removable memory card.

4. The sphygmomanometer of claim 1, wherein said memory device comprise an insertable and removable the memory card.

5. The sphygmomanometer of claim 4, wherein said memory card controls measuring device, memory card.

6. The sphygmomanometer of claim 4, further comprising and interface for connecting said memory card, wherein said control device comprises a controller and wherein said controller activates said sphygmomanometer when said memory card is connected with said interface.

7. The sphygmomanometer of claim 4, further comprising an interface for connecting said memory card, wherein said memory card comprises a controller and wherein said controller activates said sphygmomanometer when said memory card is connected with said interface.

8. The sphygmomanometer of claim 1, wherein said control device is programmable.

9. The sphygmomanometer of claim 8, wherein said control device comprises a day program for carrying out measurements during the day and a night program control for carrying out measurements at night.

10. The sphygmomanometer of claim 9, wherein operation of said sphygmomanometer during said night program generates less noise than operation of said sphygmomanometer during said day program.

11. The sphygmomanometer of claim 1, wherein said common housing comprises an input/output interface for communicating information between the user and said sphygmomanometer.

12. The sphygmomanometer of claim 1, further comprising an alarm for emitting an alarm signal before a measurement is carried out.

13. The sphygmomanometer of claim 12, wherein said alarm is capable of being programmed to activate on before day measurements.

14. The sphygmomanometer of claim 1, further comprising a sensor for determining position and movement data for said sphygmomanometer.

15. The sphygmomanometer of claim 14, wherein said position and movement data is stored in said memory device.

16. The sphygmomanometer of claim 14, wherein said position and movement data is transmitted to said control device.

17. The sphygmomanometer of claim 16 wherein said control device initiates a blood pressure measurement only when said position and movement data matches predetermined criteria.

18. The sphygmomanometer of claim 12, wherein said control devices initiates a blood pressure measurement only at the end of a predetermined time span after the alarm signal is emitted.

19. The sphygmomanometer of claim 9, further comprising a sensor for determining position and movement data for said sphygmomanometer, wherein said position and movement date is transmitted to said control device, and wherein said control device initiates either said day program or said night program based on said position and movement data.
